(19) United States
(12) Patent Application Publication
(10) Pub. No.: US 2011/0207119 A1
(43) Pub. Date: Aug. 25, 2011

(54) METHODS FOR PREDICTING A CANCER PATIENT'S RESPONSE TO SUNITINIB

(76) Inventors: Sarah L. Suchy, Pittsburgh, PA (US); Lauren M. Hancher, Pittsburgh, PA (US); Stacey L. Brower, Pittsburgh, PA (US)

(21) Appl. No.: 13/029,895

(22) Filed: Feb. 17, 2011

Related U.S. Application Data
(60) Provisional application No. 61/306,784, filed on Feb. 22, 2010.

(51) Int. Cl. C12Q 1/68; C12Q 1/02

(52) U.S. Cl. 435/6.1; 435/29

(57) ABSTRACT

The present invention provides methods for individualizing chemotherapy for cancer treatment, and particularly for evaluating a patient's responsiveness to sunitinib prior to treatment. The method comprises expanding malignant cells in culture from a patient's tumor specimen, contacting the cultured cells with one or more active agents including sunitinib, and evaluating or quantifying the response to the active agent(s). The result of the assay is a dose response curve, which may be evaluated using algorithms described herein, so as to quantitatively assess drug sensitivity. The in vitro response to the drug as determined by the method of the invention is correlative with the patient's in vivo response upon receiving sunitinib during chemotherapeutic treatment, in the course of standardized or individualized chemotherapeutic regimen.

### FIELD OF THE INVENTION

The present invention relates to individualizing cancer treatment, and particularly to individualizing cancer treatment by evaluating a patient tumor specimen for its responsiveness to sunitinib prior to treatment.

### BACKGROUND

In an attempt to individualize cancer treatment, in vitro drug-response assay systems (chemoresponse assays) have been developed to predict the potential efficacy of chemotherapy agents for a given patient prior to their administration. Such systems, which include the MTT assay and the differential staining cytotoxicity (DiSC) assay, are not considered to produce reliable results for all chemotherapeutic agents.

Sunitinib is a small molecule inhibitor that targets multiple receptor tyrosine kinases, blocking at least tumor proliferation and angiogenesis. The proteins and pathways inhibited by sunitinib include: PDGFRα, PDGFRβ, VEGFR1, VEGFR2, VEGFR3, KIT, FLT3, CSF-1R, and RET. Sunitinib is FDA-approved for the treatment of renal cell carcinoma (RCC) and gastrointestinal stromal tumors (GIST). It is currently being evaluated for clinical efficacy in other solid tumor types, such as breast.

Because the reported clinical response rate of sunitinib is low to moderate, an integrated biomarker that aids in identifying patients likely to exhibit a positive response to the drug would be a useful clinical tool.

### SUMMARY OF THE INVENTION

The present invention provides methods for individualizing chemotherapy for cancer treatment, and particularly for evaluating a patient's responsiveness to sunitinib prior to treatment.

The method generally comprises expanding malignant cells in culture from a patient's specimen (e.g., biopsy specimen), contacting the cultured cells with one or more active agents including sunitinib, and evaluating or quantifying the response to the active agent(s). In certain embodiments, monolayer(s) of malignant cells are cultured from explants prepared by mincing the tumor tissue, and the cells of the monolayer are suspended and plated for chemosensitivity testing. The result of the assay is a dose response curve, which may be evaluated using algorithms described herein, so as to quantitatively assess drug sensitivity. The in vitro response to the drug as determined by the method of the invention is correlative with the patient's in vivo response upon receiving sunitinib during chemotherapeutic treatment (e.g., in the course of standardized or individualized chemotherapeutic regimen).

In some embodiments, the patient has a solid tissue tumor, such as a renal cell carcinoma, gastrointestinal tumor (e.g., GIST), or breast cancer. As disclosed herein, studies with primary cultures of specimens demonstrate a high degree of response heterogeneity to sunitinib. When the response of 39 primary cultures of breast cancer were tested, 7.6% of cultures responded to sunitinib as a single agent, 20.5% were determined to have an intermediate response, and 71.7% were determined to be non-responsive. These results agree with the reported response rate for sunitinib in the clinical setting for breast cancer.

These results show that sunitinib efficacy can be evaluated in vitro using the cell-based chemoresponse assay disclosed herein, to determine which patients might benefit from this agent, and thereby avoiding unnecessary treatment in patients for which the drug is not efficacious.

### DESCRIPTION OF THE FIGURES

FIG. 1 shows a dose response curve for the SKOV3 cell line (ovarian carcinoma cell line) upon in vitro exposure to sunitinib.

FIG. 2 shows the dose response curves for 39 primary breast cancer specimens to in vitro sunitinib exposure.

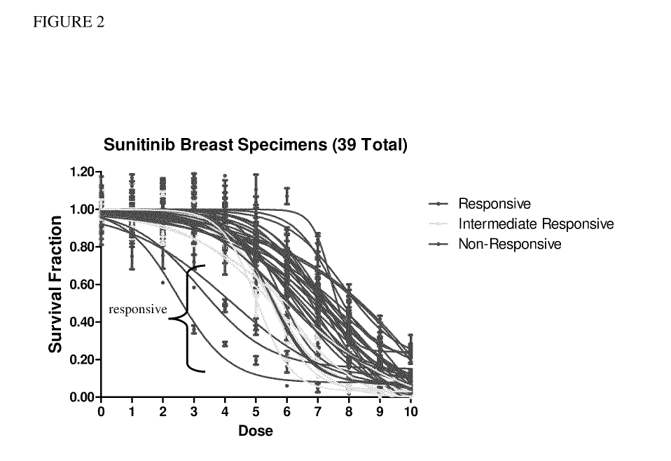

### DETAILED DESCRIPTION OF THE INVENTION

The present invention provides methods for individualizing chemotherapy for cancer treatment, and particularly, provides an in vitro chemoresponse assay for evaluating a patient's responsiveness to sunitinib prior to treatment. The method generally comprises culturing and expanding the malignant cells from a patient's specimen (e.g., biopsy), contacting the cultured cells with one or more active agents including sunitinib, and evaluating and/or quantifying the response to the drug(s). The in vitro response to the drug as determined by the method of the invention is correlative (e.g., predictive) of the patient's in vivo response upon receiving sunitinib during a chemotherapeutic regimen.

The patient generally has a cancer for which sunitinib is a candidate treatment, for example, alone or in combination with other therapy. For example, the cancer may be selected from kidney, GI, breast, lung, mesothelioma, ovarian, colorectal, endometrial, thyroid, nasopharynx, prostate, head and neck, liver, pancreas, bladder, and brain. In certain embodiments, the tumor is a solid tissue tumor and/or is epithelial in nature. For example, the cancer may be breast cancer, a renal cell carcinoma, or a gastrointestinal stromal tumor.

The present invention involves conducting chemoresponse testing with one or a panel of chemotherapeutic agents on cultured cells from the cancer patient. The panel includes sunitinib. In certain embodiments, the chemoresponse method is as described in U.S. Pat. Nos. 5,728,541, 6,900,027, 6,887,680, 6,933,129, 6,416,967, 7,112,415, 7,314,731, 7,501,260, 7,575,868, and 7,642,048 (all of which are hereby incorporated by reference in their entireties). The chemoresponse method may further employ the variations or embodiments described in US Published Patent Application Nos. 2007/0059821 and 2008/0085519, both of which are hereby incorporated by reference in their entireties.

Briefly, in certain embodiments, cohesive multicellular particulates (explants) are prepared from a patient's tumor tissue sample (e.g., a biopsy sample) using mechanical fragmentation. This mechanical fragmentation of the explant may take place in a medium substantially free of enzymes that are capable of digesting the explant. However, in some embodiments, some limited enzymatic treatment may be conducted, for example, to help reduce the size of the explants. Generally, the tissue sample is systematically minced using two sterile scalpels in a scissor-like motion, or mechanically equivalent manual or automated opposing incisor blades. This cross-cutting motion creates smooth cut edges on the resulting tissue multicellular particulates. The tumor particulates each measure from about 0.25 to about 1.5 mm³, for example, about 1 mm³.

After the tissue sample has been minced, the particles are plated in culture flasks (e.g., about 5 to 25 explants per flask). For example, about 9 explants may be plated per T-25 flask, or about 20 particulates may be plated per T-75 flask. For purposes of illustration, the explants may be evenly distributed across the bottom surface of the flask, followed by initial inversion for about 10-15 minutes. The flask may then be placed in a non-inverted position in a 37° C. CO₂ incubator for about 5-10 minutes. Flasks are checked regularly for growth and contamination. Over a period of a few days to a few weeks a cell monolayer will form. Further, it is believed (without any intention of being bound by the theory) that tumor cells grow out from the multicellular explant prior to stromal cells, such as fibroblasts and macrophages that may be initially present within the explants. Thus, by initially maintaining the tissue cells within the explant and removing the explant before the emergence of substantial numbers of stromal cells (e.g., at about 10 to about 50 percent confluency, or at about 15 to about 25 percent confluency), growth of the tumor cells (as opposed to substantial numbers of stromal cells) into a monolayer is facilitated. Further, in certain embodiments, the tumor sample or explants may be agitated to help release tumor cells from the tumor explant, and the tumor cells cultured to produce a monolayer. For example, the tumor specimen or explants may be agitated by placing the specimen or explants in a container, and shaking, rocking, or swirling the container, or striking the container against a hard surface.

The use of this procedure to form a cell culture monolayer helps maximize the growth of representative tumor cells from the tissue sample, and the resulting monolayer may comprise greater than about 60% (malignant) epithelial cells, or greater than about 70%, or greater than about 80%, or greater than about 90% (malignant) epithelial cells. The epithelial and/or malignant character of the cells may be confirmed using standard techniques. For example, see 2008/0085519, which is hereby incorporated by reference in its entirety.

Prior to the chemotherapy assay, the growth of the cells may be monitored, and data from periodic counting may be used to determine growth rates which may or may not be considered parallel to growth rates of the same cells in vivo in the patient. Monolayer growth rate and/or cellular morphology and/or epithelial character may be monitored using, for example, a phase-contrast inverted microscope. Generally, the monolayers are monitored to ensure that the cells are actively growing at the time the cells are suspended for drug exposure. Thus, the monolayers will be non-confluent when the cells are suspended for chemoresponse testing.

Generally, the agents are tested against the cultured cells using plates such as microtiter plates. For the chemosensitivity assay, a reproducible number of cells is delivered to a plurality of wells on one or more plates, preferably with an even distribution of cells throughout the wells. For example, cell suspensions are generally formed from the monolayer cells before substantial phenotypic drift of the tumor cell population occurs. The cell suspensions may be, without limitation, about 4,000 to 12,000 cells/ml, or may be about 4,000 to 9,000 cells/ml, or about 7,000 to 9,000 cells/ml. The individual wells for chemoresponse testing are inoculated with the cell suspension, with each well or “segregated site” containing about 10² to 10⁴ cells. The cells are generally cultured in the segregated sites for about 4 to about 30 hours prior to contact with an agent.

Generally, any growth media suitable for expanding the population of malignant cells may be used in connection with the invention.

Each test well is then contacted with at least one pharmaceutical agent, or a sequence of agents. At least one series of test wells is contacted with sunitinib. Sunitinib (marketed as SUTENT, and previously known as SU11248) is an oral, small-molecule, multi-targeted receptor tyrosine kinase (RTK) inhibitor that was approved by the FDA for the treatment of renal cell carcinoma (RCC) and imatinib-resistant gastrointestinal stromal tumor (GIST). Sunitinib inhibits cellular signaling by targeting multiple receptor tyrosine kinases (RTKs). These include all receptors for platelet-derived growth factor (PDGF-Rs) and vascular endothelial growth factor receptors (VEGFRs), which play a role in both tumor angiogenesis and tumor cell proliferation. The simultaneous inhibition of these targets presumably leads to both reduced tumor vascularization and cancer cell death, and ultimately tumor shrinkage. Sunitinib also inhibits KIT (CD117), the RTK that, when improperly activated by mutation, drives the majority of gastrointestinal stromal cell tumors. Sunitinib has been recommended as a second-line therapy for patients whose tumors develop mutations in KIT that make them resistant to imatinib, or who become intolerant to the drug. In addition, sunitinib inhibits other RTKs. These include: RET, CSF-1R, and flt3. The fact that sunitinib targets many different receptors, leads to many of its side effects such as the classic hand-foot syndrome, stomatitis, and other dermatologic toxicities, and could also in part be responsible for sunitinib's heterogeneous patient response rate.

In addition to Sunitinib, the panel of active agents tested may comprise at least one agent selected from a platinum-based drug, a taxane, a nitrogen mustard, a kinase inhibitor, an EGFR inhibitor (e.g., tyrosine kinase inhibitor or antibody targeting the extracellular domain), a pyrimidine analog, a podophyllotoxin, an anthracycline, and a topoisomerase I inhibitor. For example, the panel may comprise 1, 2, 3, 4, or 5 agents selected from bevacizumab, capecitabine, carboplatin, cecetuximab, cisplatin, cyclophosphamide, docetaxel, doxorubicin, epirubicin, etoposide, 5-fluorouracil, gemcitabine, irinotecan, oxaliplatin, paclitaxel, panitumumab, tamoxifen, topotecan, pemetrexed, and trastuzumab, in addition to other potential agents for treatment. In certain embodiments, the chemoresponse testing includes one or more combination treatments, such combination treatments including one or more agents described above. Generally, each agent in the panel is tested in the chemoresponse assay at a plurality of concentrations representing a range of expected extracellular fluid concentrations upon therapy.

For example, sunitinib may be tested at concentrations within the range of about 0.05 μM to about 75 μM, or from about 0.1 μM to about 56 μM. For example, sunitinib may be tested with at least 2, 3, 4, 5 or 6 doses within the range of about 0.4 μM to about 15 μM. Table 1 illustrates an exemplary range of doses for testing sunitinib in vitro in accordance with the invention.

The efficacy of each agent in the panel is determined against the patient's cultured cells, by determining the viability of the cells (e.g., number of viable cells). For example, at predetermined intervals before, simultaneously with, or beginning immediately after, contact with each agent or combination, an automated cell imaging system may take images of the cells using one or more of visible light, UV light and fluorescent light. Alternatively, the cells may be imaged after about 25 to about 200 hours of contact with each treatment (e.g., about 3 days, or about 72 hours). The cells may be imaged once or multiple times, prior to or during contact with each treatment. Of course, any method for determining the viability of the cells may be used to assess the efficacy of each treatment in vitro.

While any grading system may be employed, in certain embodiments the grading system may employ from 2 to 10 response levels, e.g., about 3, 4, or 5 response levels. For example, when using three response grades, the three grades may correspond to a responsive grade (or sensitive), an intermediate responsive grade, and a non-responsive grade (or resistant). In certain embodiments, the patient's cells show a heterogeneous response across the panel of agents, making the selection of an agent particularly crucial for the patient's treatment.

The output of the assay is a series of dose-response curves for tumor cell survivals under the pressure of a single or combination of drugs, with multiple dose settings each (e.g., ten dose settings). To better quantify the assay results, the invention employs in some embodiments a scoring algorithm accommodating a dose-response curve. Specifically, the chemoresponse data are applied to an algorithm to quantify the chemoresponse assay results by determining an adjusted area under curve (aAUC). The aAUC takes into account changes in cytotoxicity between dose points along a dose-response curve, and assigns weights relative to the degree of changes in cytotoxicity between dose points. For example, changes in cytotoxicity between dose points along a dose-response curve may be quantified by a local slope, and the local slopes weighted along the dose-response curve to emphasize cytotoxic responses.

For example, aAUC may be calculated as follows.

Step 1: Calculate Cytotoxity Index (CI) for each dose, where CI=Mean_{drug}/Mean_{control}.

Step 2: Calculate local slope (S_{d}) at each dose point, for example, as S_{d}=(CI_{d}−CI_{d-1})/Unit of Dose, or S_{d}=(CI_{d-1}−CI_{d})/Unit of Dose.

Step 3: Calculate a slope weight at each dose point, e.g., W_{d}=1−S_{d}.

Step 4: Compute aAUC, where aAUC=ΣW_{d}CI_{d}, and where, d=each dose, e.g., 1, 2, . . . , 10. Equation 4 is the summary metric of a dose response curve and may used for subsequent regression over reference outcomes.

Usually, the dose-response curves vary dramatically around middle doses, not in lower or higher dose ranges. Thus, the algorithm in some embodiments need only determine the aAUC for a middle dose range, such as for example (where from 8 to 12 doses are experimentally determined, e.g., 10 doses), the middle 4, 5, 6, or 8 doses are used to calculate aAUC. In this manner, a truncated dose-response curve might be more informative in outcome prediction by eliminating background noise.

The numerical aAUC value (e.g., test value) may then be evaluated for its effect on the patient's cells, and compared to the same metric for other drugs on the patient's cells. For example, a plurality of drugs may be tested, and aAUC determined as above for each, to determine whether the patient's cells have a sensitive response, intermediate response, or resistant response to each drug. Further, the measures may be compared to determine the most effective drug.

In some embodiments, each drug is designated as, for example, sensitive, or resistant, or intermediate, by comparing the aAUC test value to one or more cut-off values for the particular drug (e.g., representing sensitive, resistant, and/or intermediate aAUC scores, or aAUC for that drug). The cut-off values for any particular drug may be set or determined in a variety of ways, for example, by determining the distribution of a clinical outcome (as described and exemplified herein) within a range of corresponding aAUC reference scores. That is, a number of patient tumor specimens are tested for chemosenstivity/resistance (as described herein) to a particular drug prior to treatment, and aAUC quantified for each specimen. Then after clinical treatment with that drug, aAUC values that correspond to a clinical response (e.g., sensitive) and the absence of significant clinical response (e.g., resistant) are determined. Cut-off values may alternatively be determined from population response rates. For example, where a patient population is known to have a response rate of 30% for the tested drug, the cut-off values may be determined by assigning the top 30% of aAUC scores for that drug as sensitive. Further still, cut-off values may be determined by statistical measures, such as mean or median scores.

In some embodiments, the aAUC value for a sensitive designation with sunitinib is less than or equal to about 7.85 (e.g., as calculated herein). An aAUC value for a resistant designation with sunitinib is equal to or greater than about 9.22 (e.g., as calculated herein). An aAUC of between about 7.85 and about 9.22 is considered an intermediate response. In other embodiments, the aAUC scores may be a continuous scale.

### EXAMPLES

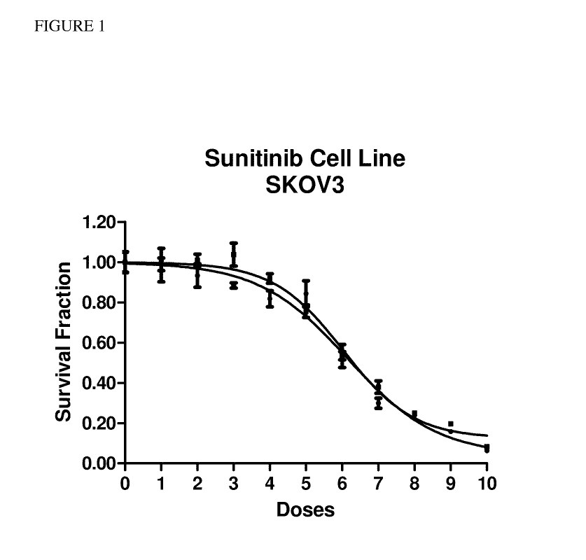

In order to characterize the performance of sunitinib in vitro, initial development was performed using the immortalized ovarian carcinoma cell line SKOV3. FIG. 1 shows a dose response curve for the SKOV3 cell line (ovarian carcinoma cell line) to sunitinib exposure in vitro, over a range of sunitinib doses shown in Table 1. The cell line SKOV3 was found to exhibit a consistent response to sunitinib.

TABLE 1

| Dose | Testing Concentration | |
|---|---|---|
| 10 | 55.3 | μM |
| 9 | 27.6 | μM |
| 8 | 13.8 | μM |
| 7 | 6.91 | μM |
| 6 | 3.46 | μM |
| 5 | 1.73 | μM |
| 4 | 0.864 | μM |
| 3 | 0.432 | μM |
| 2 | 0.216 | μM |
| 1 | 0.108 | μM |

After assay development, thirty-nine primary breast carcinoma cultures were treated with sunitinib at the doses shown in Table 1.

All specimens treated with the drug were confirmed to contain a majority of epithelial cells (>65%) via immunocytochemistry. A 10-dose range of drug concentrations was used to treat the cell line and breast specimens for 72 hours. After treatment, the cultures were fixed with ethanol, and stained with DAPI. Any cells remaining adherent after treatment were considered live, and the DAPI stained nuclei were counted. The resulting dose response curves were analyzed.

aAUC values were calculated, essentially as follows:

aAUC=Σ[1(Xᵢ₋₁−Xᵢ)]*Xᵢ

(Xᵢ is the normalized cell count of dose i)

TABLE 2
Example of calculating aAUC

| | | | | [1 − (Xᵢ₋₁ − | |
|---|---|---|---|---|---|
| Dose | Xi | Xᵢ₋₁ − Xᵢ | 1 − (Xᵢ₋₁ − Xᵢ) | Xᵢ)]*Xᵢ | |
| 0 | 1.000 | | 1.000 | | |
| 1 | 0.894 | 0.106 | 0.894 | 0.799 | |
| 2 | 0.610 | 0.284 | 0.716 | 0.437 | |
| 3 | 0.361 | 0.250 | 0.750 | 0.271 | |
| 4 | 0.284 | 0.077 | 0.923 | 0.262 | |
| 5 | 0.197 | 0.087 | 0.913 | 0.180 | |
| 6 | 0.061 | 0.136 | 0.864 | 0.053 | |
| 7 | 0.030 | 0.031 | 0.969 | 0.029 | |
| 8 | 0.019 | 0.011 | 0.989 | 0.019 | |
| 9 | 0.089 | −0.070 | 1.070 | 0.095 | |
| 10 | 0.106 | −0.017 | 1.017 | 0.108 | |
| | | | | | aAUC: 2.252 |

Example: if I=1, Xᵢ₋₁−Xᵢ=1.00−0.894=0.106

1−(Xᵢ₋₁−Xᵢ)=1−0.106=0.894

[1−(Xᵢ₋₁−Xᵢ)]*Xᵢ=0.106*0.894=0.799

aAUC=Σ[1−(Xᵢ₋₁−Xᵢ)]*Xᵢ=0.799+0.437+ . . . +0.019+0.095+0.108=2.252

Dose response curves of the 39 breast specimens revealed that 7.6% (3 of 39) were responsive to sunitinib (aAUC of ≦7.85), 20.5% (8 of 39) of specimens exhibited an intermediate response (aAUC of 7.85-9.22), and 71.7% (28 of 39) were non-responsive to the drug (aAUC of ≧9.22).

The data collected from the primary breast carcinoma cultures and cell line were consistent when compared to the reported clinical response rate of sunitinib in breast cancer. The invention can thus contribute to the current chemotherapy identification and selection process for oncologists and their patients.

All references cited herein, including all patent and non-patent literature, are hereby incorporated by reference for all purposes.

1. A method for predicting the efficacy of sunitinib for a cancer patient, comprising:
culturing malignant cells from a tumor specimen from the patient;
contacting the cultured cells with sunitinib; and
evaluating the response of the malignant cells to sunitinib.

2. The method of claim 1, wherein the cancer is selected from a renal cell carcinoma, gastrointestinal stromal tumor, or breast cancer.

3. The method of claim 1, wherein the cancer is breast cancer.

4. The method of claim 1, wherein the malignant cells are cultured from a plurality of tumor explants in a monolayer culture.

5. The method of claim 4, wherein the tumor explants are prepared by mechanical fragmentation of the patient's tumor specimen.

6. The method of claim 5, wherein the tumor specimen is minced.

7. The method of claim 6, wherein the explants measure from about 0.25 to about 1.5 mm³.

8. The method of claim 4, where the monolayer cells are suspended in culture media, and the cells plated for chemosensitivity testing.

9. The method of claim 8, wherein at least 5 dilutions of sunitinib are added across a plurality of wells within the range of about 0.05 μM to about 75 μM.

10. The method of claim 8, wherein from 2 to 6 doses of sunitnib are added across a plurality of wells within the range of about 0.4 μM to about 15 μM.

11. The method of claim 1, wherein sunitinib is contacted with the cells for about 3 days, and then cell viability quantified.

12. The method of claim 11, wherein cell viability is quantified by visible light, UV light, or fluorescent light.

13. The method of claim 12, wherein cells are stained with DAPI.

14. The method of claim 1, wherein the response of the cells to sunitinib is evaluated by preparing a dose response curve, and determining an adjusted AUC (aAUC).

15. The method of claim 14, wherein the aAUC assigns weights relative to the degree of change in cytotoxicity between dose points.

16. The method of claim 15, wherein changes in cytotoxicity between dose points are quantified by a local slope, and the local slopes weighted along the dose-response curve to emphasize cytotoxic responses.

17. The method of claim 16, wherein aAUC is calculated by:
calculating a Cytotoxity Index (CI) for each dose;
calculating a local slope (S_{d}) at each dose point;
calculating a slope weight at each dose point, by W_{d}=1−S_{d}; and
calculating aAUC, where aAUC=ΣW_{d}CI_{d}, and where d represents each dose in a range.

18. The method of claim 17, wherein aAUC is calculated for a truncated dose response curve.

19. The method of claim 17, further comprising, assigning the tumor sample as being responsive, non-responsive, or intermediate responsive to sunitinib.
